(19) United States
(12) Patent Application Publication
(10) Pub. No.: US 2025/0295517 A1
(43) Pub. Date: Sep. 25, 2025

(54) EXTERNAL MALE CATHETER

(71) Applicant: Swistle IP, LLC, Minneapolis, MN (US)

(72) Inventors: Melissa Fryberger, Minneapolis, MN (US); David Gregerson, Lawrenceville, GA (US); Zyad Kadri, Meridian, ID (US)

(73) Assignee: Swistle IP, LLC, Minneapolis, MN (US)

(21) Appl. No.: 19/085,858

(22) Filed: Mar. 20, 2025

Related U.S. Application Data
(60) Provisional application No. 63/567,707, filed on Mar. 20, 2024.
(60) Provisional application No. 63/635,253, filed on Apr. 17, 2024.
(60) Provisional application No. 63/757,755, filed on Feb. 12, 2025.

(51) Int. Cl. A61F 5/453; A61F 5/44

(52) U.S. Cl. CPC A61F 5/453; A61F 5/4408

(57) ABSTRACT

What is disclosed in some embodiments is an improved external male catheter that includes a tubular sheath with an opening therethrough. The sheath has an insertion end and a urine tube adapter end, where the opening at the urine tube adapter end is sized and shaped to attach to a urine drainage tube. The catheter also includes a collar comprised of a stiffer material than the tubular sheath. The collar is fixedly attached to the insertion end of the tubular sheath. The collar is shaped as a ring with an internal circumference and an external circumference, wherein the external circumference of the collar is smaller than a circumference of the opening on the tubular sheath at the insertion end.

### CROSS REFERENCE TO RELATED APPLICATIONS

The present application claims priority to U.S. Provisional Application Ser. No. 63/567,707, filed Mar. 20, 2024, entitled, “External Male Catheter;” U.S. Provisional Application Ser. No. 63/635,253, filed Apr. 17, 2024, entitled, “External Male Catheter;” and U.S. Provisional Application Ser. No. 63/757,755, filed Feb. 12, 2025, entitled, “External Male Catheter;” the contents of each of which are hereby incorporated into the present application in their entirety.

### FIELD OF THE INVENTION

Some embodiments of the present disclosure are directed to improved urine collection devices. More particularly, the present disclosure in some embodiments is directed to improved external male catheters.

### BACKGROUND

Generally speaking, catheters are devices that collect urine. Catheters may be needed for a variety of reasons. For example, catheters may be needed for individuals with bladder problems, including problems with the muscles controlling the bladder. Catheters may also be used by people with problems with the nerves that control their bladder; people with urinary retention issues; during some types of bladder tests and treatments; during surgery; and people who are very unwell in the hospital may also need catheters.

Depending on why the catheter is needed, catheters may be needed for long-term use, for example with people that have urinary problems. But more typically, catheters are only needed for a short period of time, or a relatively short period of time. Short-term indwelling catheters are passed through the urethra but remain in place for only a short period of time, for example, during surgery. Long-term indwelling catheters may also be passed through the urethra but remain in place for a longer period of time. These catheters need to be changed regularly. In contrast, intermittent catheters may be used at regular intervals when the bladder needs to be emptied. The catheter is then removed. Depending on the catheter and the circumstances of the user, some people are able to do this themselves, which is referred to as self-catheterization. Unlike these catheters that are inserted into the urethra, an external catheter is placed over the penis to collect urine. As with intermittent catheters, depending on the device and the circumstances of the user, some people are able to self-catheterize.

Problems with catheters are well-known and can cause people that need to use them pain, discomfort, inconvenience, and embarrassment. Catheters can leak, become blocked, and/or fall out or fall off, resulting in additional pain, discomfort, inconvenience and/or embarrassment for catheter users. Further, indwelling catheters can cause urinary tract infections. External catheters are typically held on the penis with adhesive tape or other pressure sensitive adhesive means. This style of catheter is hard to apply and can cause pain and injury. Such catheters also have a tendency to leak. In addition, user error with applying external catheters is a significant problem for both medical staff and patients. Even when applied correctly, external catheters can cause discomfort, skin inflammation and irritation, and sores on the head and shaft of the penis. They can also be difficult and/or painful to remove and can leave adhesive behind on the skin which can cause irritation. They may also fail in an hour or less. With all of these issues with existing external catheters it is difficult for able bodied/minded people apply, but nearly, if not completely impossible for people with physical and/or mental limitations to apply, such as for example, people with strokes, the use of only one hand or arm, etc. Accordingly, a need exists for a catheter that minimizes these disadvantages of catheters. Specifically, a need exists for an improved external catheter that allows users to self-catheterize relatively easily to minimize pain and discomfort from indwelling catheters and to minimize or eliminate embarrassment due to health care professionals and/or family or friends needing to put on or help put on an external catheter. More specifically, an improved external catheter is needed that allows individuals in particularly compromised physical and/or mental and/or emotional conditions to self-catheterize.

### BRIEF SUMMARY OF THE INVENTION

What is disclosed in some embodiments of the present disclosure is an improved external male catheter that includes a tubular sheath with an opening therethrough. The sheath has an insertion end and a urine tube adapter end, where the opening at the urine tube adapter end is sized and shaped to attach to a urine drainage tube. The catheter also includes a collar comprised of a more stiff material than the tubular sheath. The collar is fixedly attached at the insertion end of the tubular sheath. The collar is shaped as a ring with an internal circumference and an external circumference, wherein the external circumference is smaller than a circumference of the opening on the tubular sheath at the insertion end. The improved external male catheter may include a collar comprised of silicone that substantially holds its shape when pressure is applied to the collar. The collar may be fixedly attached to the insertion end of the tubular sheath by adhesive tape. In some embodiments, the collar may be fixedly attached to the insertion end of the tubular sheath by a heat-seal adhesive.

The improved external male catheter may also include an internal wrap. The internal wrap may be shaped like a ring. The internal wrap may be fixedly attached to the collar, wherein the internal wrap may be threaded through the internal circumference of the collar and folded over a portion of the external circumference of the collar. The internal wrap may have a length greater than that of the collar. The internal wrap may be comprised of a thin, flexible material. In some embodiments, the folded over portion of the internal wrap may be adhered to the portion of the external circumference of the collar by tape. In some embodiments, the folded over portion of the internal wrap may be adhered to the portion of the external circumference of the collar by a heat-seal adhesive.

In some embodiments, the improved external male catheter may also include a ring-shaped pad with a circumference smaller than the internal circumference of the collar, wherein the ring-shaped pad is fitted inside the internal circumference of the collar. The ring-shaped pad may be secured to the internal circumference of the collar by an adhesive.

The improved external male catheter in some embodiments may also include a stopper that is sized and shaped to snugly fit into the opening at the urine tube adapter end of the tubular sheath.

The present disclosure in some embodiments may include a collar for securing to a part of the penis, the collar may include a ring, the ring may have an interior circumferential surface and an exterior circumferential surface, and a tubular-shaped internal wrap attached to the collar by threading the internal wrap through the interior circumferential surface of the collar and folding a portion of the internal wrap over the exterior circumferential surface of the collar. In some embodiments, the folded-over portion of the internal wrap may be secured to the exterior circumferential surface of the collar by an adhesive. In some embodiments the adhesive may be tape. In some embodiments the adhesive may be a heat-seal adhesive. The collar may be comprised of silicone. The internal wrap may be comprised of nitrile.

In some embodiments of the present disclosure, a method of self-catheterization is provided. The method includes applying lubricant to a portion of a wearer's penis, grasping an improved catheter by a collar portion of the catheter, while holding the collar portion of the improved catheter, inserting the head of the penis through an opening at the collar portion of the catheter until the collar portion of the catheter sits at a base of the penis. In some embodiments, the method includes using only one hand to grasp and hold the collar portion during self-catheterization. In some embodiments, the collar may be comprised of silicone.

### BRIEF DESCRIPTION OF THE DRAWINGS

The following drawings are illustrative of embodiments and do not limit the scope of the invention. The drawings are not necessarily to scale and are intended for use in conjunction with the following detailed description. Embodiments of the invention will be described with reference to the drawings, in which numerals may represent like elements in some cases only.

FIG. 1a is a perspective view of an embodiment of a tubular sheath of an improved external catheter, according to some embodiments of the present disclosure;

FIG. 1b is a sectional view of one embodiment of a collar for an improved external catheter, according to some embodiments of the present disclosure;

FIG. 2 is a sectional view of an external catheter including the tubular sheath of FIG. 1a and the collar of FIG. 1b, in accordance with some embodiments of the present disclosure.

FIG. 3a is an exploded view of one embodiment of the present disclosure.

FIG. 3b is a sectional view of the embodiment shown in FIG. 3a.

FIG. 4 is an exploded partial view of one embodiment of the present disclosure.

FIG. 5 is a partial perspective view of the embodiment shown in FIG. 4, according to some embodiments of the present disclosure.

FIG. 6 is top side view of one embodiment of the present disclosure.

FIG. 7 is a bottom side view of the embodiment shown in FIG. 6, according to some embodiments of the present disclosure.

FIG. 8a is a perspective view of one embodiment of an improved external male catheter shown with the retainer section in an unrolled position, according to some embodiments of the present disclosure.

FIG. 8b is a perspective view of the embodiment of an improved external male catheter shown in FIG. 8a with the retainer section in a rolled position, according to some embodiments of the present disclosure.

FIG. 8c is a perspective side view of the embodiment of a securing component for an improved external male catheter, according to some embodiments of the present disclosure.

FIG. 8d is a perspective bottom side view of the embodiment of a securing component shown in FIG. 8c, according to some embodiments of the present disclosure.

FIG. 8e is a perspective side view of the embodiment of a securing component for an improved external male catheter as shown in FIGS. 8c and 8d with the internal wrap pulled out from the inside of the collar, according to some embodiments of the present disclosure.

FIG. 8f is a perspective view of an embodiment of an improved external male catheter in a collapsed state, according to some embodiments of the present disclosure.

FIG. 9 is a perspective view of one embodiment of an improved male catheter, according to some embodiments of the present disclosure.

FIG. 10 is a perspective view of one embodiment of an improved male catheter, according to some embodiments of the present disclosure.

FIG. 11 is a perspective view of one embodiment of an improved male catheter, according to some embodiments of the present disclosure.

FIG. 12 is a schematic showing dimensions for one embodiment of an improved male catheter, according to some embodiments of the present disclosure.

FIG. 13a is a perspective view of one embodiment of a tab ring for use with some embodiments of the present disclosure.

FIG. 13b is a perspective view of another embodiment of a tab ring for use with some embodiments of the present disclosure.

FIG. 14a is a perspective view of the tab ring of FIG. 13a in use with one embodiment of the device, in accordance with some embodiments of the present disclosure.

FIG. 14b is a perspective view of the tab ring of FIG. 13b in use with one embodiment of the device, in accordance with some embodiments of the present disclosure.

FIG. 15a is a top side view of an embodiment of a half-moon shelf in use with an embodiment of an improved male catheter, in accordance with some embodiments of the present disclosure.

FIG. 15b is a bottom side view of an embodiment of the half-moon shelf of FIG. 15a in use with an embodiment of an improved male catheter, in accordance with some embodiments of the present disclosure.

FIG. 16 is a perspective view of an embodiment of a duckbill tab in use with an embodiment of an improved male catheter, in accordance with some embodiments of the present disclosure.

FIG. 17 is a perspective view of an embodiment of a retainer hole in use with an embodiment of an improved male catheter, in accordance with some embodiments of the present disclosure.

FIG. 18 is a partial exploded view of an embodiment of a stopper for use with an embodiment of an improved male catheter, in accordance with some embodiments of the present disclosure.

FIG. 19 is a partial perspective view of an embodiment of a stopper with a lead for use with an embodiment of an improved male catheter, in accordance with some embodiments of the present disclosure.

### DETAILED DESCRIPTION OF THE INVENTION

The present disclosure is directed in some embodiments to an improved external male catheter that may be more easily applied, and in some cases, significantly more easily applied than existing devices. Further, in some embodiments, the external male catheter may have fewer and, in some cases, no adverse consequences for the wearer than existing devices, including but not limited to, irritation of the skin, and/or pain, and/or slippage or unwanted movement of the device during use, and/or seepage of urine onto the skin of the wearer.

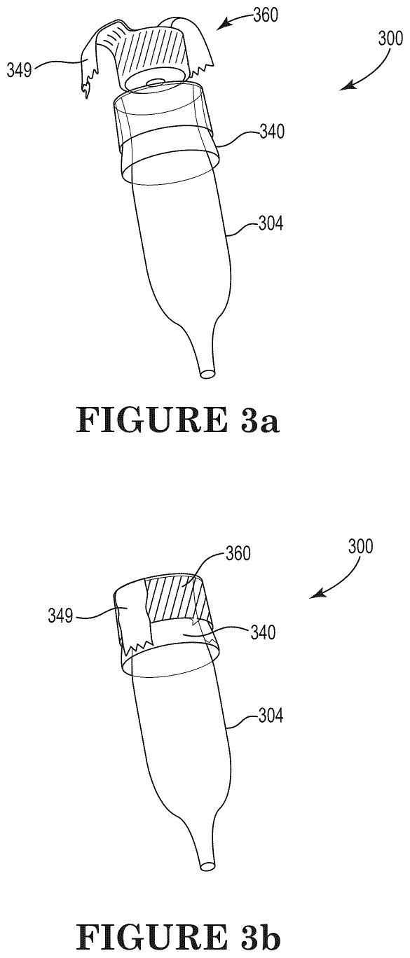

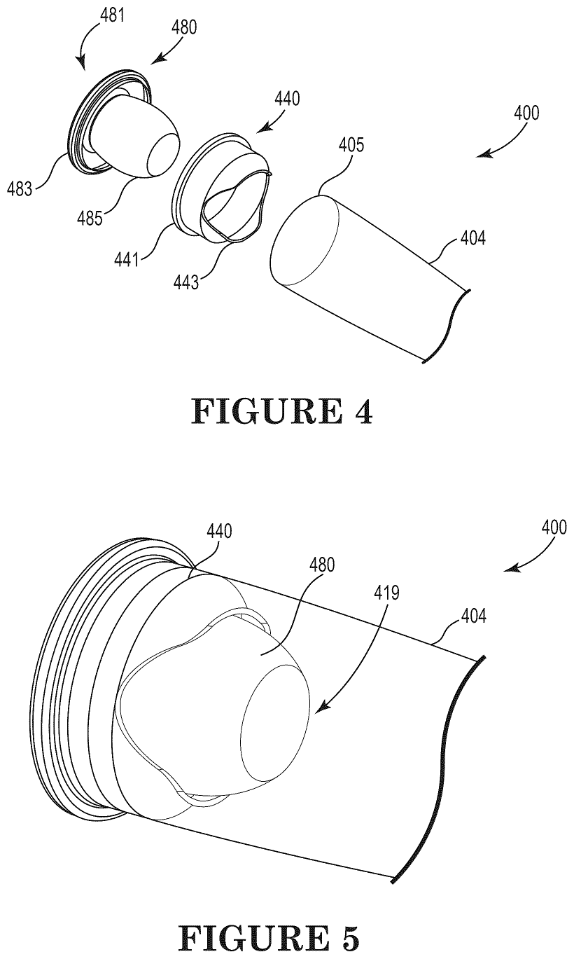

As shown in FIG. 2, the device 100 may include a penis cover or sheath 104. In some embodiments, device 100 may also include a collar or grip 140 that may be coupled to the sheath 104, as shown in FIG. 2. In still other embodiments, the device 300 may also include a pad 360 secured within the interior circumference of the collar 340, as shown in FIGS. 3a and 3b. In still other embodiments, the device 400 may include a wrap or internal wrap 480 that may be secured to the collar 440 and the length of the wrap 480 may extend beyond the length of the collar 440 to form a skin-hugging wrap around the penis, or a portion of the penis, as shown in FIGS. 4 and 5.

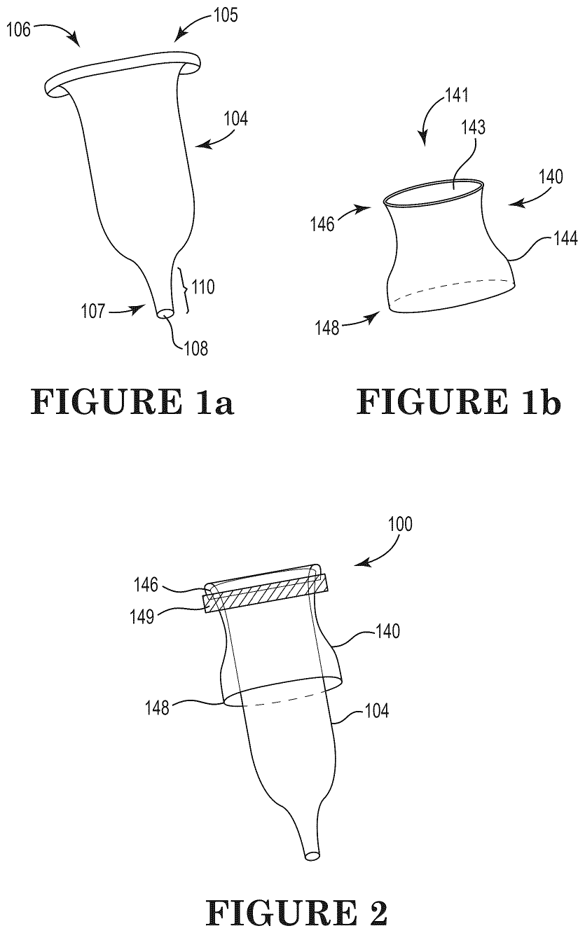

With reference back to Figure la, the sheath 104 may be a unitary penis cover, as is shown in FIG. 1, or it may be comprised of one or more sections coupled together. The sheath 104 may include an insertion end 105 and a drainage end or drainage tube adapter end 107. The insertion end 105 of the device may have an opening 106 through which the penis may be inserted, similar to that of a condom. The drainage end 107 of the sheath 104 may have a drainage opening 108 for urine to leave the device. In some embodiments, the drainage end 107 may include a drainage tube adapter 110 that may couple to a urine drainage device that includes a collector for removing urine from the device (not shown). The drainage tube adapter 110 may be comprised of the same material and be unitary with the entirety of the sheath, while in other embodiments, the drainage tube adapter, or some portion thereof may be a separate piece of material secured to, or fitted into, for example, the sheath. In some embodiments, the drainage tube adapter 110 may be sized and shaped to couple to standard and known urine drainage device tubes. It will be understood, however, that the drainage tube adapter may be configured to accommodate a differently sized and/or shaped drainage device tube.

The sheath 104 may be comprised of any suitable, flexible, non-irritating, and/or hypoallergenic material, or combination of materials. In some embodiments, the sheath 104 may be comprised of, but not be limited to, for example silicone, polypropylene (PP), polyethylene (PE), or any combination thereof. The sheath 104 may have any suitable shape, including circumference and/or length. The shape of the device should be suitable for use with a majority of users. In some embodiments, different sizes, i.e., circumferences and/or lengths of devices may accommodate differently sized wearers, for example adult wearers versus child wearers. The length of the sheath 104 running from the insertion end 105 to the drainage end 107 in some embodiments may be long enough to completely cover the entirety of the penis and then extend beyond the head of the penis some distance in order for urine to collect and drain toward the drainage tube adapter portion 110 of the sheath. The circumference of the sheath 104 may be uniform in some embodiments, while in other embodiments the circumference may vary at one or more points along the length of the sheath. For example, the circumference of the sheath may narrow, or may significantly narrow at the drainage tube adapter portion 110 of the sheath, as shown in FIG. 1. In some embodiments, the circumference of the sheath may vary or also vary along the length of the sheath at one or more points other than the drainage tube adapter portion.

The sheath may have any suitable thickness(es). The thickness of the material of the sheath may be uniform throughout the entirety of the sheath, or the thickness of the material may vary at different points on the sheath. For example, the thickness of the sheath material may be thicker at the drainage tube adapter portion 110 in order to make connection to the drainage device tube easier and/or more secure. Similarly, the thickness of the sheath 104 material may be thicker at the insertion end 105. In some embodiments, the thickness of the sheath, or some portion of the sheath may be similar to that of a condom. However, in some embodiments, the thickness may be greater and in some cases several times greater than that of a condom to provide more strength and structure to the device, which may allow for easier application and/or wear. In some embodiments, the thickness of the sheath may allow the sheath when worn to stay apart from the skin of the penis, i.e., to hold its shape away from the skin of the penis.

In some embodiments the inside and/or outside and/or a portion of the inside and/or outside of the penis cover 104 may include a coating or other application to provide one or more benefits, for example, to sterilize the penis cover, prevent infections, bacterial or other mold growth, to help the penis cover 104 at the insertion end 105 adhere to the penis shaft, and/or to allow the penis cover 104 to be more easily applied.

In some embodiments, the sheath 104 may be uniformly smooth on the interior and exterior. In other embodiments, the exterior and/or interior of the sheath 104 may include texture at some or all portions of the sheath 104. Adding texture to some or all of the exterior of the sheath 104 may help the user more securely grip the sheath during application. Adding texture to some or all of the interior of the sheath may help the sheath stay in place at portions where the sheath may contact the skin and/or may help direct urine away from the penis and toward the drainage tube adapter 110. The texture in some embodiments may be any suitable texture, for example, channels, ribs, dots, rings, and/or any other texture or combination of textures or features.

In some embodiments, the entirety of the sheath 104 may be clear, and/or generally translucent, such that one can see from the exterior of the sheath into the interior of the sheath, which may help ensure that the device is properly placed on the penis. In other embodiments, the entirety of the sheath 104 may be opaque, such that one cannot see from the exterior of the device into the interior of the device, which may help provide privacy to the wearer during use. In still other embodiments, the sheath 104 may have some portions that are translucent and some portions that are opaque. The sheath 104 may be colored or clear in color. In colored embodiments, the sheath 104 may be uniformly colored or the color may vary and/or change at different parts of the sheath 104. Adding color to the sheath 104 material may help people with poor eyesight more easily find, apply and/or adjust the device. Alternatively, adding colored pigment to the sheath material may make use of the device more enjoyable. In some embodiments, patterns, words, numbers, and/or objects or pictures may be imprinted on, or added to the sheath.

In some embodiments, the external male catheter 100 may also include a collar or grip 140, as shown in FIGS. 1b and 2, for example. The grip 140 in some embodiments may generally form a ring with an opening 141 through the center. The collar 140 may be fixedly or detachably secured to the penis cover at the insertion end 105 of the penis cover 104. When attached, the collar 140 may provide a relatively stable, solid surface that may be readily handled by a user to permit easy application of the improved external male catheter 100.

In some embodiments, the collar 140 may be comprised of a relatively solid, but in some embodiments still somewhat flexible material, such as but not limited to silicone, any other plastic, or any other suitable plastic, other material, or combination of materials, for example that provides a handling surface for the user to direct application of the catheter 100 over the penis. The collar 140 may be generally a ring shape, with an opening 141 through the middle, an inner circumference 143, and an outer circumference 144. The collar 140 may have an upper end 146 and a lower end 148. The collar 140 may also have a length that runs from the upper end 146 of the collar 140 to the lower end 148 of the collar 140. In some embodiments, the collar 140 may be sized and shaped to accommodate the penis cover 104 within the circumference of the collar, but also sized such that the penis cover 104 may be folded over the exterior of the collar 140 as shown in FIG. 2.

The outer circumference 144 of the collar 140 in some embodiments may be uniform from the upper end 146 to the lower end 148. In still other embodiments, the outer circumference 144 may vary along the length of the collar 140. Similarly, the inner circumference 143 of the collar 140 may be uniform from the upper end 146 to the lower end 148, or the circumference 143 may vary along the length of the collar 140 along with or differently from the outer circumference. The exterior surface of the collar 140 may be smooth across the entire exterior surface in some embodiments. In other embodiments, the exterior of the collar 140 may have one or more surface features, such as rings, ribs, nubs, protrusions, indentations, and/or any other kind of surface feature or combination of features to help a user grip the collar 140 to aid in application, for example. It will be understood, however, that surface features may have other benefits, as well. Similarly, the interior surface of the collar 140 may also be entirely generally smooth, or it may have one or more surface features. The length of the collar 140 may be uniform along the circumference of the collar in some embodiments. In other embodiments, the length of the collar 140 may vary along the circumference.

In some embodiments, the collar 140 may be made of a clear material(s) so that the penis may be viewable through the collar 140. In other embodiments, the collar 140 may be made of an opaque material(s) that cannot be seen through or may only be partially seen through. The material or materials comprising the collar 140 may include pigments that color the collar 140. In some embodiments, the collar 140 may have a design, pattern, letters, words, numbers, etc., and/or any combination thereof. Such markings and/or colors, or combination thereof may help users identify the collar 140 and therefore the correct end of the device to insert the penis into, for example. Instructions and/or other messages may also be provided on the collar 140 in visible writing and/or braille for example. Writing or other markings may be provided in UV light, or glow in the dark markings, in some embodiments.

The collar 140 may be adhered to the insertion end 105 of the penis cover 104 by any suitable means. In some embodiments, the collar 140 may be detachably secured to the penis cover 104, such that the collar 140 may be able to be cleaned and reused with a new penis cover 104. In other embodiments, the collar 140 may be fixedly attached to the penis cover in order to prevent reuse of the collar, for example. In some embodiments, adhesive tape 149 may be used to secure the collar 140 to the sheath 104, as shown in FIG. 2. In other embodiments, an applied adhesive material may be used, including, but not limited to a heat-sealed adhesive. In still other embodiments a combination of securing means may be used.

In one embodiment, the sheath 104 may be thread through the opening 141 in the collar 140 from the lower end of the collar through the upper end of the collar. The insertion end 105 of the sheath 104 (or top end of the sheath) may then be folded over the exterior of the collar 140, as shown in FIG. 2. The portion of the sheath 104 folded over the exterior of the collar 140 may be secured to the collar by an applied adhesive, such as a heat-sealed adhesive in some embodiments. In other embodiments, the sheath may be secured to the collar by tape 149. In still other embodiments, both an adhesive and tape may be used, and/or any other suitable means, or combination of means of attaching the sheath to the collar may be used.

As may be seen in FIGS. 3a and 3b, in an embodiment of a device 300 that includes a pad 360, the pad 360 may have tape 349 attached, such that when the pad 360 is inserted into the collar 340 within the sheath 304, the tape 349 on the pad 360 may hold all of the components securely together, i.e., the sheath 304, collar 340, and pad 360. Of course, it will be recognized that other manners of securing the pad to the collar and/or sheath may be used.

In other embodiments, as shown for example, in FIGS. 4 and 5, the sheath 404 may not be thread through the collar 440 and folded over the collar 440, but instead the collar 440 may be attached to the insertion end 405 of the sheath 404, by friction fit, heat-seal adhesive, tape, and/or any other suitable method, as may be seen for example. In still other embodiments, the collar may be adhered to the interior of the sheath further down the length of the sheath from the insertion end. In such embodiments, the collar could be adhered to the inside of the sheath by double-sided tape, heat-seal adhesive, surface features on the interior of the sheath and/or the exterior of the collar, and/or by any other suitable means, or combination of means.

In some embodiments, the collar 340 may include a friction pad 360 on the interior circumferential surface of collar 340, as shown in FIGS. 3a and 3b. Like the collar 340, the friction pad 360 may be ring-shaped and have a circumference that is smaller, or only slightly smaller than the circumference of the collar 340, such that the pad 360 may fit inside of the collar 340. The pad 360 may cover all or only a portion of the interior circumferential surface of the collar 340. The friction pad 360 may be comprised of any suitable material that may help the pad 360 (and hence the device) to grip to the skin. In some embodiments, heat from the user's skin may help the skin grip to the pad material, thereby keeping the device in place. For example, the friction pad may be comprised of silicone in some embodiments, while in other embodiments it may be comprised of any other suitable material or combination of materials, including any other suitable plastic, woven, or non-woven material. The pad may be secured to the collar by any suitable means, including but not limited to an adhesive, such as tape, or heat-seal adhesive and/or any other means or combination of means.

In some embodiments, the improved external male catheter may include an interior/internal wrap or sheath. For example, one embodiment of a device 400 with an internal wrap 480 is shown in FIGS. 4-7, which will be discussed in more detail further below. In embodiments with an internal wrap, the internal wrap, like the collar may have a tubular, or ring-shaped geometry, forming an interior and exterior circumference, and with an upper end and a lower end, whereby a length of the internal wrap is defined between the upper and lower ends of the wrap. The wrap may be comprised of any suitable material or combination of materials. In some embodiments, the internal wrap may be comprised of a plastic, rubber, or other balloon-like material in some embodiments, that has both flexibility and some strength, for example. In some embodiments, for example, the wrap may be comprised of any suitable pliable material that may create a diaphragm, including but not limited to latex, and/or nitrile.

The wrap may be clear, opaque, have any color, surface feature, and/or design, for example. In some embodiments, the wrap may be secured to the collar of the device at the upper end of the wrap with the lower end of the wrap extending below the length of the collar at the lower end of the collar. In use, the internal wrap may envelop a portion of the penis, whereby the head of the penis may protrude through the lower end of the wrap, but much of the rest of the penis is held by the wrap, which forms a continuous seal with the penis, thereby keeping the device in place, and keeping urine from escaping out of the insertion end of the device. Seals may also be formed at each end of the wrap, i.e., at the upper end of the wrap and the lower end of the wrap. Because of the material comprising the wrap, despite the snug fit of the wrap on the penis, the penis is still able to move and change shape freely and easily with the material of the wrap conforming to such changes.

The wrap may be secured to the collar in any suitable way, including by tape, adhesive, both, or any other way of securing the internal wrap to the collar, and/or the pad, and/or the sheath, for example. In some embodiments, the wrap may be threaded through the inside of the collar and folded over the collar to form a snug seal against the exterior of the collar. The wrap may have a smaller circumference than the interior circumference of the collar such that a diaphragm may be formed at the upper end of the wrap that is attached to the collar.

In some embodiments the improved external male catheter may include an absorbent pad. The absorbent pad may include a circular, ring-shaped pad that may fit inside the penis cover rolled over the collar, inside of the collar, and/or inside of the friction pad. In some embodiments, the absorbent pad may be comprised of a non-woven material. In other embodiments, the absorbent pad may be comprised of a woven material, and/or any absorbent material or combination of materials may be used that may help prevent or minimize urine exiting the device from the insertion end of the device and/or aid in making application of the device easier, more comfortable, and/or aid in retaining the device in place on the penis. In some embodiments the adhesive pad may be secured to the device with adhesive tape. In other embodiments, the adhesive pad may be secured to one or more portions of the device by an adhesive, glue, and/or any other suitable method or combination of methods.

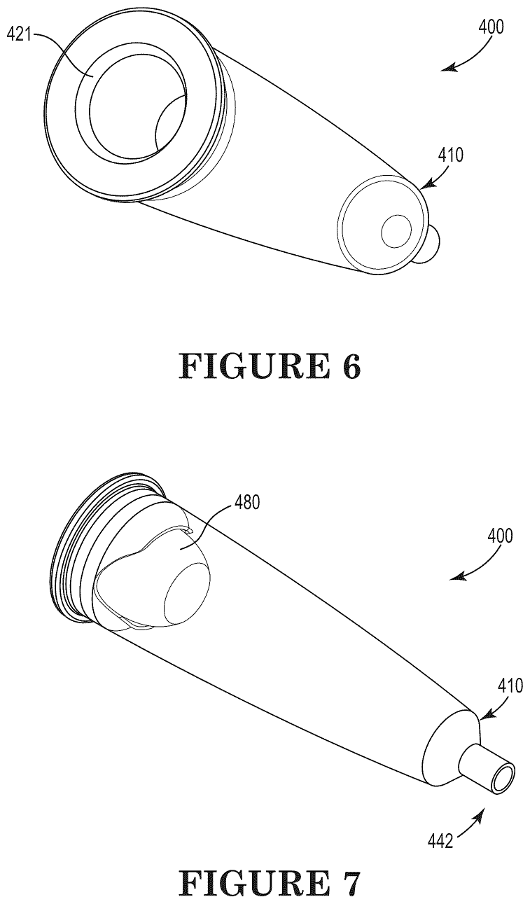

FIGS. 4-7 show an embodiment of an improved external catheter 400 that includes an internal wrap 480. As may be seen in the exploded view of FIG. 4, the embodiment 400 may include a sheath 404, a collar 440, and an internal wrap 480. Embodiments may also include a urine tube adapter section 410, as shown in FIGS. 6-7.

The internal wrap 480 in some embodiments may be comprised of a relatively thin and/or medium thickness, flexible material, such as latex, or nitrile, for example. The wrap 480 may be generally tubular or ring-shaped with an opening 481 therethrough. The wrap 480 may have an internal circumference and an external circumference, an upper end 483 and a lower end 485, with a length of the wrap being defined by the distance between the upper 483 and lower ends 485 of the wrap 480. In some embodiments, the circumference of the internal wrap 480 at the upper end 483 may have a greater width between the inner circumference than the outer circumference, thereby creating a diaphragm, which may be seen in FIG. 4. As may be seen in FIG. 5, the internal wrap 480 may be secured to the collar 440, which once coupled, may be then secured to the sheath 404. The internal wrap 480 may be secured to the collar 440 by any suitable means, including, but not limited to friction fit or any other type of fit, tape, and/or tape, and/or adhesive. The collar 440 coupled with the internal wrap 480 may then be secured to the sheath 404 by any suitable means, including, but not limited to friction fit or any other fit, tape, and/or adhesive for example. It will be understood that the internal wrap 480 may also directly secure to the sheath 404 in some embodiments, while in some embodiments, only the internal wrap 480 may secure to sheath 404, while the collar 440 only secures to the internal wrap 480.

With reference to FIGS. 6 and 7, during application of the device 400 on the penis, the penis may be inserted first through the opening 481 in the internal wrap 480 covering the collar 440 at the top end 483 of the wrap 480, and then through the lower end 485 of the wrap 480, such that the head of the penis exits through the lower end 485 of the wrap 480. The wrap 480 creates two seals on the penis. The first seal 421 is shown in FIG. 6. The first seal 421 is created at the base of the penis. The second seal 419 is shown in FIG. 5. The second seal 419 is created above the head of the penis. The two seals 419, 421 permit a change of size of the penis without losing the seal while the catheter is worn.

In some embodiments, the device 400 may also include a collar/grip 440 that may be comprised of a relatively hard material, such as plastic or silicone, for example, as discussed further above and below. The collar 440 may be used to aid the user in applying the catheter 400. In some embodiments, the collar 440 does not directly contact the penis. The wrap 480 may be thread through the inside of the collar 440 and folded over the top of the collar 440, in some embodiments. The wrap 480 may be attached to the collar 440 by adhesive in some embodiments, and/or tape, and/or any other suitable means or combination of means. The adhesive may not contact the penis in some embodiments. The collar 440 has a first end 441 and a second end 443. The first end 441 is the end that is closest to the insertion end 405 of the sheath 404, once assembled. The second end 443 of the collar 440 is the end that is closest to the urine exit end 442 of the sheath 404. The wrap 480 in some embodiments may be attached to the exterior of the first end 441 of the collar 440. In some embodiments, the second end 443 of the collar 440 may include a wave-like shape, that in some embodiments may create two longer u-shaped sections, and two concave, shorter sections. This shape may allow the penis to naturally contract and expand without breaking the seal on the wrap. The length of the wrap 480 may be longer than the length of the collar 440, as shown in FIG. 7. It will be understood, however, that other embodiments of the internal wrap and/or collar may include any shape and/or design or other feature as is described herein. For example, in some embodiments, the collar may not have varying length, etc.

Finally, in some embodiments, a urine tube adapter 410 may be included, as shown in FIGS. 6 and 7. The urine tube adapter 410 in some embodiments may be unitary with the sheath 404, while in other embodiments, the urine tube adapter 410 may be comprised of different material(s) from the sheath 404, whereby the urine tube adapter section 410 may be securely affixed or coupled to the sheath 404. In some embodiments, the urine tube adapter section 410 may be comprised of a plastic material and may be configured to securely connect to a urine tube at a urine exit end 442 of the adapter 410 for collection of the urine from the catheter 400. The adapter 410 may be connected to the sheath 404 by an adhesive, heat-seal adhesive, tape, and/or any other suitable method in embodiment where the adapter 410 is not unitary with the sheath 404.

In use, embodiments of the device may be packaged in a collapsed state, which may be permitted by the flexible nature of the sheath. In other embodiments, the device may be packaged in a non-collapsed state. If the components of the device are already connected, the user proceeds directly to placement of the catheter on the penis. In embodiments where components need to be connected, the user first connects the components and then proceeds to placement.

The user may first extend the device into its open state if it was packaged in a collapsed state. The user may grab the collar or the grip of the device and insert the head of the penis into the device opening at the insertion end of the device until the device is in place up to the shaft of the penis. In embodiments of devices with internal wraps, the head of the penis is inserted through the lower end of the internal wrap. Notably, in some embodiments, the user may apply the device with a single hand as a result of the solidity of the collar/grip, thereby permitting people with only one hand or arm to be able to self-catheterize. In some embodiments, the user may apply a lubricant, such as Vaseline for example, or any other suitable lubricant to some or all parts of the penis prior to application of the device. In some embodiments, the user may use a shimmy action to help apply the device over the penis.

In embodiments with an absorbent pad, the pad may help provide slip action during application. In order to adjust the device, the head of the penis may be pulled to seat the collar/grip at the maximum height to achieve optimal placement. Once application is complete, in embodiments with an absorbent pad, the absorbent pad may provide a soft friction hold as skin moisture causes subtle expansion. In embodiments with an internal wrap, the internal wrap creates seals at the top end and bottom end of the internal wrap to provide a secure, but flexible hold. A urine drain tube may then be connected to the opening in the urine exit end of the penis cover. At this point, the catheter is ready to be used.

In some embodiments, the improved external catheter would be completely assembled as a unitary device that may be packaged for use, such that the user need only open the package and apply the catheter. In other embodiments, one or more components may be packaged in an unconnected manner, such that the user may connect the components prior to application.

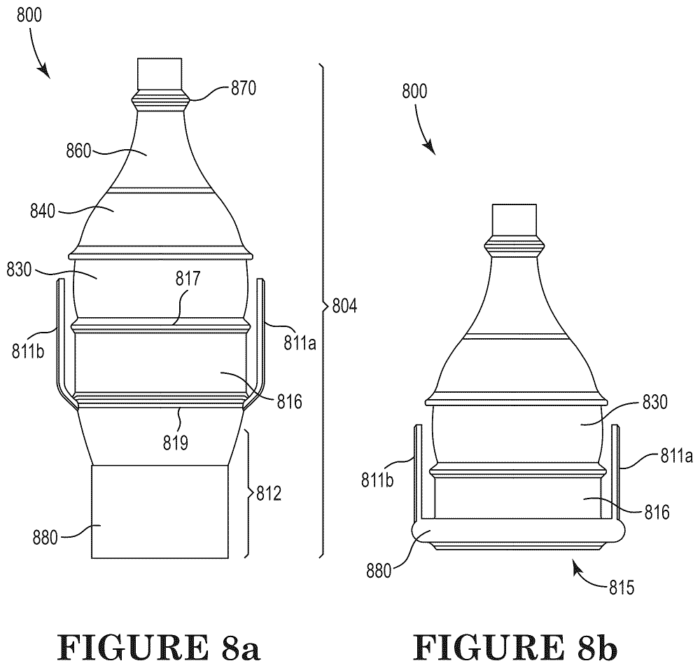

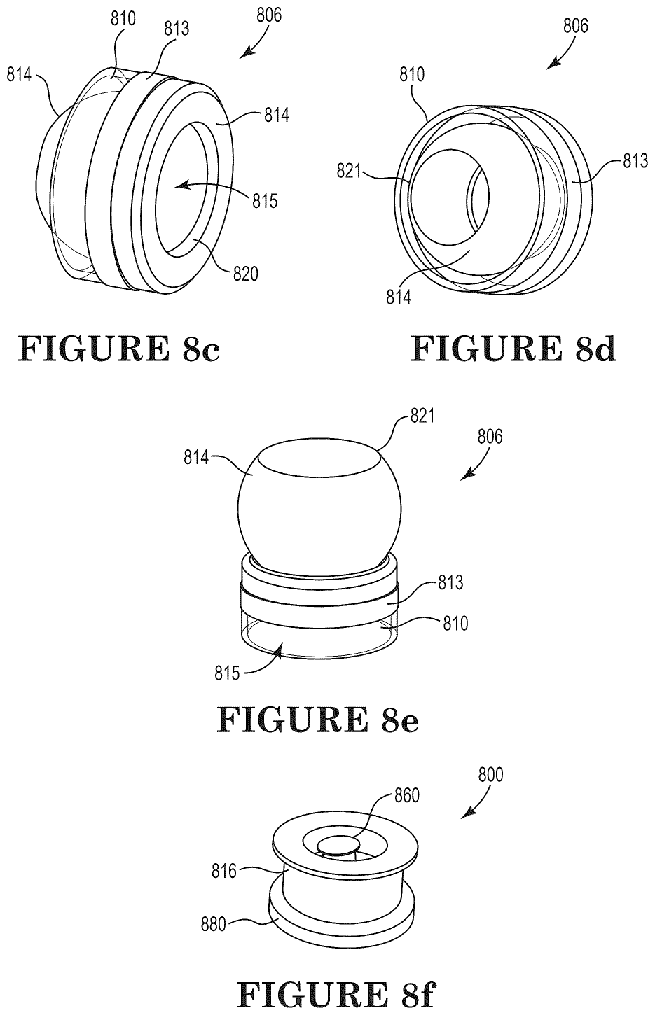

In another embodiment of the present disclosure, as may be seen in FIGS. 8 a-f, an external male catheter device 800 may comprise a unitary sheath 804 that includes a securing component 806 that is coupled to the inside of the unitary sheath 804.

The unitary sheath may be formed or molded from any suitable process as a single piece. Though it will be understood that the unitary sheath may in other embodiments be comprised of multiple sections that are secured together. Unitary sheath may be comprised of any suitable material, or combination of materials, including, but not limited to, for example, silicone, polyurethane, polypropylene (PP), polyethylene (PE), or any other suitable flexible non-irritating material or combination of materials.

Unitary sheath may have a plurality of different sections that may have one or more functions and/or different functions. Each section, or one or more sections may have a different size and/or shape than one or more other sections of the unitary sheath. While certain shapes may be described herein and shown in the figures, it will be understood that other sizes and shapes than those shown and described for each section are within the spirit and scope of the present disclosure. Further, while a certain number of sections are described and shown, it will be understood that more or fewer sections of the unitary sheath are also within the spirit and scope of the present disclosure.

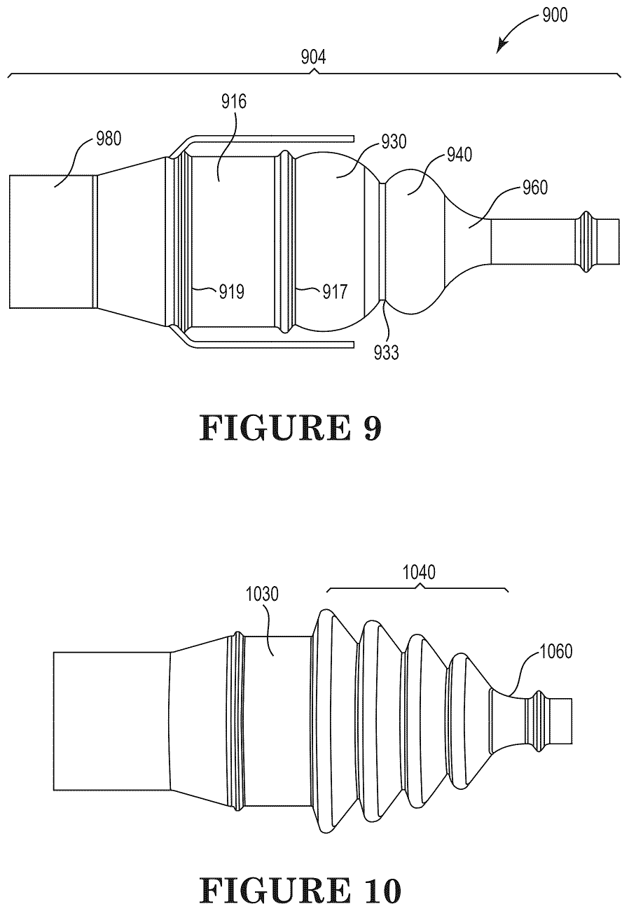

With reference to FIG. 8a, in some embodiments, the unitary sheath 804 may include a securing component section 816; a housing (or penis housing) section 830; a urine collection section 840; a urine drain tube connector section 860; and a retainer section 880. As may be seen in FIG. 8a, the plurality of sections may have differing lengths, and different circumferences, and sometimes changing circumferences within a section. While an alternate number, size, and/or shape of sections comprising the unitary sheath are within the spirit and scope of the invention, in some embodiments, sections with differing sizes and/or shapes may provide improved pliability of the device, and/or case of movement once the device is applied, allowing for a secure retention of the device as/when the penis changes size and shape. For example, FIG. 9 shows another embodiment of a device 900 that includes a unitary sheath 904 having differently sized and/or shaped sections, including, differently sized and/or shaped securing component section 916; housing section 930; urine collection section 940; urine drain tube connector section 960; and retainer section 980 than those shown in FIG. 8a.

With reference back to FIG. 8a, securing component section 816 may provide several functions. Securing component section 816 includes securing component 806, which is internally coupled to unitary sheath 804. Securing component 806, shown independently when it is outside of unitary sheath 804 in FIGS. 8c-e, includes collar grip 810 and internal wrap 814. FIG. 8c shows the entrance view where the penis is inserted into opening 815. FIG. 8d shows a bottom view of the securing component 806. FIG. 8e shows a side view of the securing component with the internal wrap 814 pulled out of the collar grip 810.

Collar grip 810 may be comprised of a generally hard and/or rigid or semi-rigid material, such as plastic, or other suitable material or combination of materials or alloys that permits a user to firmly grip the securing component section 816 of the device 800 when the securing component 806 is secured within the unitary sheath 804. In some embodiments for example, collar grip 810 may be comprised of a thermoplastic material, such as polycarbonate or Acrylonitrile Butadiene Styrene (ABS), though it will be understood that any suitable material, or combination of materials that have the desired properties of sturdiness may be used. As may be seen, collar grip 810 may generally be a ring with an opening 815 therein. In some embodiments, the length of collar grip 810 ring may be dimensioned to allow a user's thumb and one or two fingers to easily grasp the collar grip 810 when it is inside of unitary sheath 804. In some embodiments, collar grip 810 may be sized such that the thumb and forefinger of the same hand may be positioned over the collar grip 810 (enclosed within securing component 816 section of the unitary sheath 804) during application, while the middle finger of the same hand may also be positioned on collar grip 810 (within securing component section 816) or housing section 830. Configuring collar grip 810 to have such a length may allow the user to more easily apply the device, particularly where the user may only have one hand available for application.

In some embodiments, internal wrap 814 of securing component 806 may be a unitary length of tubular material that is pliable and non-irritating to the skin. For example, in some embodiments, the internal wrap 814 may be comprised of a thin, flexible, elastic plastic such as, but not limited to latex and/or nitrile. As may be seen in FIGS. 8c and 8d, internal wrap 814 may be positioned inside of collar grip 810, with the top portion of internal wrap 814 being folded up and over the top portion of the collar grip 810. When internal wrap 814 is secured to collar grip 810, the clastic nature of internal wrap 814 forms flexible opening 815. Flexible opening 815 can be used as a seal against the skin of the penis when the device 800 is fully positioned on the shaft of the penis. The elastic nature of opening 815 can conform to various diameters, shapes and contours of the penis shaft. The opposite end 821 of internal wrap 814 may be a cut or formed opening that may be positioned further down the shaft and may reside on or behind the penis head. Internal wrap end 821 forms an additional seal against the skin of the penis located on or behind the penis head. The region between the seals on the internal wrap 814 can also grip the shaft of the penis. In some embodiments internal wrap 814 may be secured to the outside of collar grip 810 by a piece of adhesive tape 813 applied over the top edge of internal wrap 814 that is folded over the outside of collar grip 810. In other embodiments, an adhesive may be applied between the collar grip 810 and the portion of the internal wrap that is folded over the collar grip 810. Any other suitable method of securing internal wrap 814 to collar grip 810 may also be used.

Securing component 806, as shown in FIGS. 8c-d, may be coupled to the interior of unitary sheath 804 in some embodiments by inserting securing component 806 into unitary sheath 804 and holding securing component 806 in place at securing component section 816 of unitary sheath 804 with an adhesive to seal securing component 806 to the inside of unitary sheath 804. Other embodiments may include any other means of holding securing component in place in unitary sheath at the securing component section thereof.

Because of the solid, generally hard material that the collar grip 810 is comprised of, it provides a firm grasping surface for the user to hold as the device 800 is applied to the penis. In some embodiments, the securing component section 816 may be delineated linearly by a bottom lip 817 and a top lip 819. Lips 817 and/or 819 may provide the user with a tactile guide to where the securing component section 816 is located on the device 800. Lip 819 may also provide a parking space for rolled retainer ring 880 (described further below).

Additionally, securing component section 816 may provide solidity for device 800 during application. As will be described further below in more detail, to secure the device 800 onto the penis, the penis is inserted through opening 815 in the securing component 806 which is accessible in the device 800 when the retainer ring 880 is in a rolled position, as shown in FIG. 8b. The solidity of collar grip 810 provides a solid boundary through which to insert the penis. When the penis is gently pushed through the opening 815, internal wrap 814 lightly but snugly hugs the shaft of the penis. The head of the penis extends through the lower end of the internal wrap 814. Accordingly, internal wrap 814 provides two seal points for the device 800 on the penis, one seal is created at the lower end 821 of the internal wrap 814 on or just above the head of the penis, and another seal is created at the upper end 820 of the internal wrap at the top of the collar grip 810. As such, the securing component 806 may act as a force multiplier that provides retention for the device during urination.

It will be understood that internal wrap 814 may be of any length, though in some embodiments may have a length that permits the head of the penis to protrude through the lower end 821 of the internal wrap 814. FIG. 8e shows internal wrap 814 pulled outside of collar grip 810. As may be seen, in some embodiments, the circumference of internal wrap may vary from one end of the internal wrap 814 to the other end. It will be understood however, that internal wrap may have any suitable shape, circumference, or length, including varying dimensions throughout the length of internal wrap.

In one embodiment no adhesive of any kind is needed to secure the device onto the penis. Instead, the seals provided by the configuration of the device securely maintain the device on the penis. This may be a significant improvement over many prior art catheters that require adhesives to be applied to at least some portion of the penis, which may cause irritation, pulling, rubbing, and/or other issues.

In some embodiments, however, an adhesive, and in some embodiments only a minimal amount of light adhesive may be used to secure the internal wrap to the penis, as needed. Examples of adhesives that may be used include, for example, but are not limited to any adhesive that may be suitable for use on the skin that will not result in, or will minimize irritation or injury to the skin. For example, in some embodiments, the adhesive may be a mild acrylic adhesive. In some embodiments, an adhesive may already be applied to internal wrap, while in other embodiments, the user may apply an adhesive prior to placement and/or repositioning.

Penis housing section 830 may generally be the section of device 800 that houses the penis, which in embodiments using a generally clear material for the unitary sheath 804, may permit a portion of the penis to be seen through housing section 830 to help determine proper placement of the device 800. The housed penis may be partially covered in internal wrap 814, with the head of the penis extending through the lower end opening 821 of internal wrap 814.

Urine collection section 840 may generally be a section of the device 800 that may help direct urine away from the penis housing section 830 and into urine drain tube connector section 860. As may be seen, in some embodiments, housing section 830 may have a generally wider circumference than urine collection section 840.

In other embodiments, as shown in FIG. 9, housing section 930 and urine collection section 940 may have significantly different shapes, including wherein the transition between the differing circumferences of the sections 930, 940 may be delineated with a pinch point 933. This geometry may help direct urine out of housing section 930—away from the penis and into urine collection section 940.

Further, in some embodiments, as may be seen in FIG. 10, urine collection section 1040 may be configured in an accordion-like fashion. As may be seen, urine collection section 1040 includes a plurality of concentric rings. Although four concentric rings are shown, it will be understood that the number of rings may be any suitable number of rings. The circumference of each ring may be the same or different, and the length of each ring may be the same or different depending on the embodiment. It will also be understood that while FIG. 10 shows an accordion configuration for the urine collection section, an accordion configuration may be used in any one or more of the sections of the device. The accordion structure may help reduce or prevent kinking where the urine drain tube connector section 1060 connects to the housing section 1030, thereby reducing, significantly reducing, or eliminating the potential for urine backflow and/or blockage. The accordion structure may also encourage urine flow toward the urine drain tube connector section and out of the device. In addition, this structure also makes it easier to collapse the device for packaging, as shown in FIG. 8f, and discussed further below.

With reference back to FIG. 8a, urine drain tube connector section 860 may be configured to secure to a urine drain tube of standard size; though urine drain tube connector section could be sized to fit any existing or after-arising drain tube assembly that helps remove urine from the device. As may also be seen, urine drain tube connector section 860 includes holder ring 870, which helps to keep section 860 circular and in the optimal position for connecting the urine drain tube connector section 860 to the drain tube. Holder ring 870 may include a plurality of concentric rings that provide a thickened area on the unitary sheath 804 to help connect the drain tube more easily. It will be understood that holder ring 870 may be part of unitary sheath 804 and/or may include a ring that may be applied and/or secured to urine drain tube connector section 860.

Retainer section 880 in some embodiments may include two positions, a rolled position as shown in FIG. 8b, and an unrolled position, as shown in FIG. 8a. As may be appreciated, the material 812 of retainer section 880 is either rolled up into the rolled position as shown in FIG. 8b, or the material 812 is unrolled, as is shown in FIG. 8a. The device 800 is applied to the penis when the retainer section 880 is in the rolled position, as shown in FIG. 8b. After the penis is inserted in opening 815 as shown in FIG. 8b, when the retainer section 880 is in the rolled position, the user then causes the retainer section 880 to transition from the rolled position shown in FIG. 8b to the unrolled position shown in FIG. 8a. Once in the unrolled position, the material 812 of the retainer section 880 surrounds the base of the shaft of the penis near the pubic bone, thereby creating a third seal for the device 800. This third seal, in addition to the two seals (820, 821) created by the internal wrap 814 of the securing component 806, provides a robust adherence between the penis and the device 800, making unwanted slippage of the device or removal of the device unlikely.

In some embodiments, a user may transition the retainer section from the rolled position to the unrolled position via the use of a pulley 811a and/or 811b attached to the device, as shown in FIGS. 8a-b.

In some embodiments, the device may include one or more pulleys that may help the user transition the retainer section 880 from the rolled to the unrolled position. The pulleys 811a-b may be integrally formed with the unitary sheath 804 or may be affixed to the device in any suitable manner, including by an adhesive, for example. In still other embodiments one or more pulleys may be simply rolled up in retainer material 812 and not be adhered to unitary sheath. In some embodiments, there may only be a single pulley, while in other embodiments (as shown in FIGS. 8a-b) there may be two pulleys, while in still other embodiments there may be more than two pulleys. In embodiments with more than one pulley, the multiple pulleys may be similarly or differently sized and/or shaped. Pulleys 811a-b may have any suitable dimensions, or shape, including but not limited to a loop, a tab (as shown in FIGS. 8a-b), or any other shape and/or size. In some embodiments, the pulleys 811 may include gripping geometries to make it easier to grab and pull. In some embodiments, the pulleys may include raised or indented markings, for example to indicate that the user has located one or more pulley 811a or 811b, which can aid in the use of the one or more pulleys 811a-b. In some embodiments, the markings may be in braille, but in other embodiments the markings may be any tactile feature.

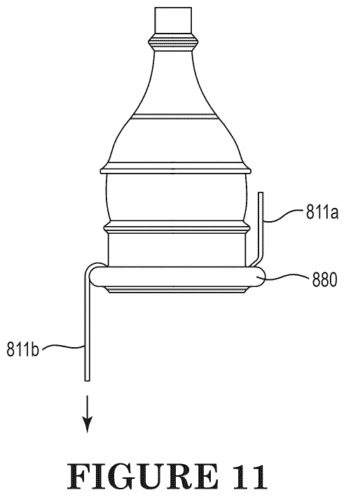

As may be seen in FIG. 8a, pulley 811 may be positioned at generally the place where the retainer section 880 and securing component section 816 meet, such that when retainer section 880 is in a rolled position (as shown in FIG. 8b), pulleys 811a-b may be partially rolled up in rolled retainer section 880. Thus, as shown in FIG. 11, the user need only pull pulley 811 upward toward the base of the penis, as is shown with pulley 811b, to transition retainer section 880 from the rolled position to the unrolled position. It will be understood that pulleys 811a-b may be placed at any point on the retainer section 880 that allows the retainer section to transition from the rolled position to the unrolled position when the user pulls on one or more pulley 811a-b. In some embodiments, the tapered geometrical design and elastic nature of retainer section 880 promotes the automatic unrolling of retainer section 880 when one or more pulleys are pulled, and/or when the rolled material of retainer section 880 is flicked from the rolled to unrolled position.

In some embodiments, the pulley may include other beneficial features. For example, in one embodiment, a clip may be attached or attachable to the free end of the pulley. In such embodiments, the clip that is clipped to the pulley may also be clipped or secured to an article that may help keep the device in place. For example, a safety pin may be attached to the free end of the pulley once the device has been completely applied. The safety pin may then be pinned through the user's hospital gown or other clothing that may help keep the device in place. It will be understood that any securing mechanism may be used, such as any pin or clip that would not harm the user.

In another embodiment, the user may transition the retainer section from the rolled to the unrolled position without the use of the pulley (though one or more pulleys may still be present in some embodiments, while in other embodiments, there may be no pulley). As may be seen in FIG. 8b, retainer section 880 is in the rolled position, as it would be after the penis is initially inserted into the device. At this point in the application process, the device will look like FIG. 8b from the perspective of the wearer. The thumb may be positioned below the retainer section 880 with the material 812 in the rolled position. With the same hand, the user may grasp the collar of securing component section 816 firmly and use his thumb to push or flick the rolled material 812 of the retention section 880 upward toward the wearer's body. This movement results in the rolled material 812 of the retainer section 880 extending upward toward the base of the penis into the unrolled position of the retainer section 880 as shown in FIG. 8a, thereby forming another retention fit seal on the penis of the user. As may be appreciated, this embodiment only requires the user to use one hand to transition the retainer section 880 from the rolled to the unrolled position. This may be very beneficial for patients that only have the use of one hand.

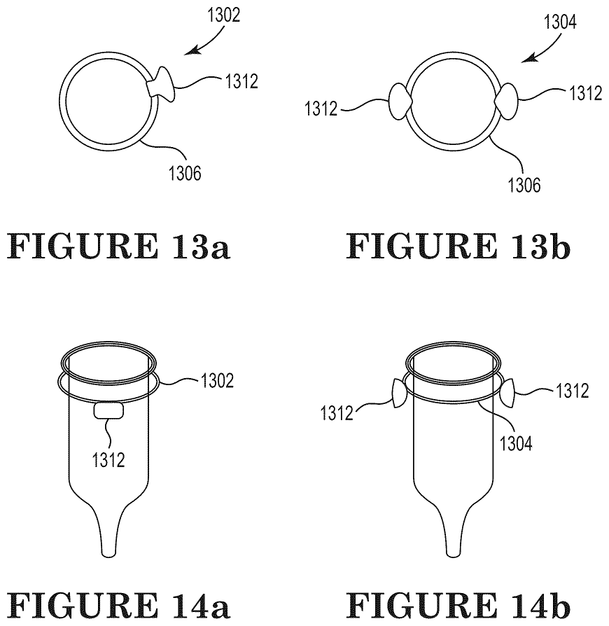

In still another embodiment that does not have a pulley, a tab ring may be used in conjunction with the device. As may be seen in FIGS. 13a and 13b, a tab ring 1302, 1304 may include a generally circular ring portion 1306 and one or more tabs 1312 attached to the ring portion 1306. As may be seen, FIG. 13a shows a tab ring with one tab, and FIG. 13b shows a tab ring with two rings. In some embodiments, the ring portion 1306 may be comprised of a flexible plastic, for example, but not limited to polypropylene. The one or more tabs 1312 may be unitary with the ring 1306 that may be manufactured as a single unit. As such, the tab(s) 1312 would also be comprised of the same material as the ring 1306. In other embodiments, the tab(s) 1312 may be comprised of a different material than the ring 1306. The tab(s) 1312 may then be secured to the ring 1306 by any suitable means, including heat sealing adhesive, tape, and/or any other suitable method. The tab(s) 1312 may have any suitable shape or geometry such that it makes it easy to use the fingers to pull on the tabs. In embodiments with multiple tabs, the tabs may have the same size and/or shape or they may have different sizes and/or shapes. The ring 1306 may have a circumference that allows it to fit relatively snugly over the catheter device up to the bottom of the retainer section when the retainer section is in the rolled position. The circumference of the ring 1306 may further be configured to be smaller than the circumference of the rolled section of the retainer.

The ring 1306 may have any desired thickness that helps in the use of the ring. The one or more tabs 1312 may have any suitable size and shape that helps a user grasp the one or more tables by the fingers. While FIGS. 13a and 13b show only a single tab and a two tab design (respectively), it will be understood that any number of tabs may be included. In use, the tab ring may already be applied to the device below the rolled portion of the retainer section. In other embodiments, the tab ring may be separate from the device, such that once the device is applied to the penis, then the user slips the tab ring over the device up to the point of rolled retainer ring. FIG. 14a shows the one-tab tab ring 1302 in position to be pulled upward, while FIG. 14b shows the two-tab tab ring 1304 in position to be pulled upward. The user may grasp the tab(s) 1312 with the forefinger and the middle finger and pull upward in the direction of the belly button to transition the retainer section from the rolled position to the unrolled position. In the two-tab tab ring design 104, the user may use both hands, one hand for each tab to pull upward to transition the retainer section from the rolled to the unrolled position.

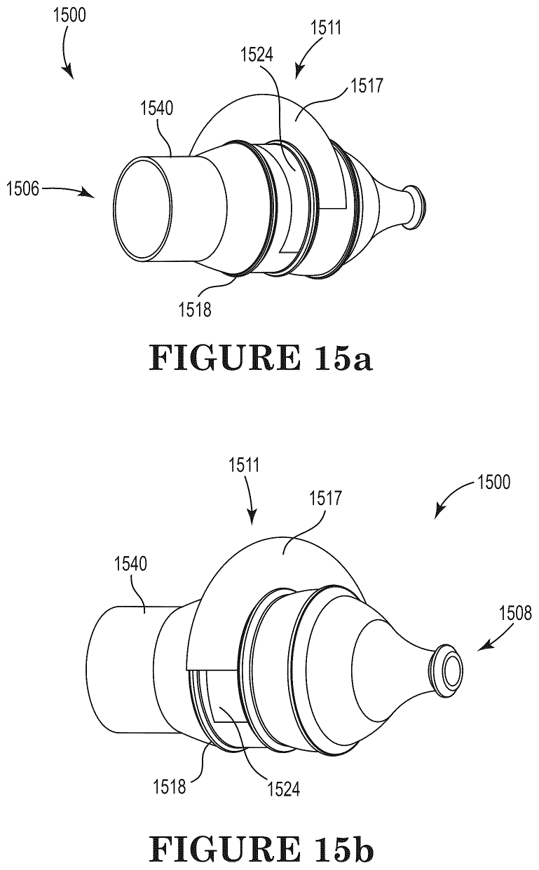

In another embodiment, as shown in FIGS. 15a and 15b, the catheter 1500 may include a shelf, and in some embodiments, a half-moon shaped and configured shelf, as shown in FIG. 15a from the retainer end 1506 of the device 1500, and in FIG. 15b from the urine exit end 1508 of the device 1500. The retainer 1540 is shown in the unrolled position. As may be seen the half-moon shelf 1511 is positioned below retainer lip 1518 that helps secure the retainer when it is in the rolled position. When the retainer 1540 is in the rolled position, the shelf 1511, situated below the rolled retainer may be pushed upward toward the belly button of the wearer to transition the retainer from the rolled position to the unrolled position. The shelf 1511 may be comprised of the same material as the sheath and may be unitarily constructed with the unitary sheath. In other embodiments, the shelf 1511 may be attached or secured to the sheath. The shelf 1511 may be comprised of the same or different material as the sheath in such embodiments. In some embodiments, the shelf 1511 may be comprised of a slightly more stiff material than the sheath, which may also help keep the sheath in place. The shelf may be a half-moon that circumscribes only a portion of the circumference of the sheath, as is shown in FIGS. 15a and 15b. In other embodiments, the shelf may circumscribe the entirety of the circumference of the sheath. In some embodiments, the shelf 1511 may include a claw portion 1524 and/or a grasping portion 1517. The grasping portion 1517, as may be seen, may have a greater width than the claw portion 1524 that may secure to the sheath. Similarly, the claw portion 1524 circumference may extend further than the grasping portion 1517 in some embodiments.

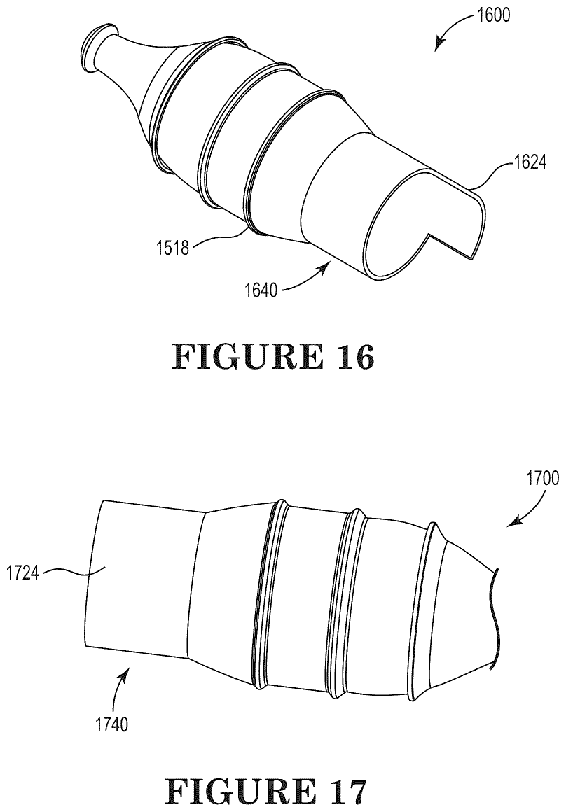

In another embodiment, shown in FIG. 16, the catheter 1600 may include a duckbill tab 1624 that may aid in transitioning the retainer section from a folded down position to an extended position. FIG. 16 shows the catheter 1600 with the retainer section in the extended or raised position. Instead of the retainer section 1640 being rolled down to the retainer holding lip 1518, the retainer section 1640 may be folded over onto the exterior of the device 1600. The retainer section in some embodiments, may be held down in the folded position by friction-fit, surface features, and or by a tape and/or light adhesive, for example, such that when the user pulls the duckbill tab 1624 upwards toward the wearer's belly button, the retainer section 1640 transitions from the folded position to the extended position. In embodiments using tape and/or an adhesive to keep the retainer section in the folded position, the tape and/or adhesive may be sufficiently strong to keep the retainer in the folded position until the duckbill tab 1624 is pulled upward, at which point the adhesive releases and/or the tape breaks allowing the retainer to extend. In embodiments that have an adhesive or other application applied to the interior surface of the retainer section, when the retainer section is in the folded-over position, which may then expose the applied material added to the interior of the retainer section, a protective wrap may surround the exposed interior section of the folded-over retainer section. In this manner, the applied material may stay in tact and undisturbed. Prior to transitioning the retainer section from the folded position to the extended position, the protective wrap may be removed. The protective wrap may be of any suitable material or combination of materials, such as a paper-based, woven, or non-woven material, fabric, rubber or any other suitable material or combination of materials. The protective wrap may be secured by an adhesive, a light adhesive, double-sided tape, or any other suitable means, or combination of means.

In some embodiments, the duckbill tab may be removable, such that when the retainer section is transitioned to the extended position, it may be removed to avoid any discomfort to the wearer. In such embodiments, for example, the duckbill tab may have a perforated attachment area such that it may simply be easily ripped off after extending the retainer portion. Alternately, the duckbill tab, may simply be cut off or otherwise removed from the device.

In yet another embodiment, shown in FIG. 17, the device 1700 may include a hole 1724 in the retainer section 1740. FIG. 17 shows the retainer 1740 in the unrolled position. A string or other gripping device may be secured, tied, or otherwise affixed through the hole 1724. When the retainer 1740 is in the rolled position, the string may hang outside of the rolled material of the retainer section 1740, allowing the user to grab onto the string. When the user pulls the string upward toward the belly button of the wearer, the retainer section 1740 transitions from the rolled position to the unrolled position. In another embodiment, the retainer section may not transition from a rolled to an unrolled position, but rather from a folded-over to an extended position. In such embodiments, the string may fall outside of the folded-over portion of the retainer section when the retainer section is in a folded position. The user may simply pull the exposed string upward toward the wearer's bellybutton to transition the retainer section to the extended position.

In some embodiments, no adhesive is needed of any kind to secure the material of the retainer section in the unrolled position to the penis. As stated above with regard to the internal wrap, this may be an advantage over some existing catheters that require the use of an adhesive to secure the catheter to the penis.

However, in alternate embodiments, an adhesive may be applied to the interior side of material of the retainer section so that when it is rolled onto the penis, it may securely adhere to the skin of the penis. Examples of adhesives that may be used include, for example, but are not limited to any adhesive that may be suitable for use against the skin that will not result in, or may minimize irritation or injury to the skin. For example, in some embodiments, the adhesive may be a mild acrylic adhesive. While embodiments are disclosed with reference to application using only one hand, of course it will be understood that two hands may also be used for application.

Device 800 may be collapsed into a packaging state, as a result of the inventive geometry of the device. As may be seen in FIG. 8f, in a packaging state, retainer section 880 is in the rolled position. Housing section 830, urine collection section 840, and a portion of urine tube connector section 860 may collapse inside of securing component section 816, leaving a portion of urine tube adapter section 860 extending above securing component section 816. Urine drain tube connector section 860 may be bent sideways and compressed on the inside wall of securing component section 816, in some embodiments. The collapsibility of the device 800 makes packaging more space efficient, and thus more cost-effective, as well as providing protection for the collapsed portions of the device during transit and storage. It will be understood that in other embodiments, the device may be packaged and/or stored in a non-collapsed state.

In use, in some embodiments, including ones where the device is collapsed for packaging, a user may open the packaging containing the device and remove the device in its collapsed state, such as is shown in FIG. 8f. The user may expand the device into an application state, by grabbing onto the portion of the urine tube connector section that extends beyond the securing component section and pulling outwardly. This motion will result in the device extending to the position shown in FIG. 8b. This is the state the device is in when the user applies the device to the penis. Prior to application, the user may apply lubricant to the head of the penis to maybe halfway up the shaft of the penis to make application easier. In some embodiments, Vaseline may be applied to the penis, though it will be understood that any suitable lubricant may be used, for example, any suitable water-based lubricant or lotion in some embodiments. In some embodiments, only the head of the penis is lightly coated with lubricant, but in other embodiments, as much or as little may be applied as needed to case application. Applying a lubricant to the penis prior to application of the device may protect the skin from any negative effects of lingering urine, for example. Lubrication may be applied by the user or an assistant prior to applying the device, or in some embodiments, lubricant may already be applied to the inside of internal wrap prior to packaging

The device as shown in FIG. 8b may then be applied to the penis, whereby the user may grasp the device at the securing component section 816 with one hand in some embodiments, whereby the thumb of one hand may be placed on one side of the collar of securing component section 816 and the pointer finger and middle finger of the same hand may grasp the securing component 806 of securing component section 816 on the other side of securing component 806. The user may then move the lubricated penis into and through the opening 815 in the securing component 806.

In some embodiments the unitary sheath may be clear and permit the user to see the head of the penis emerge through opening 821 of internal wrap 814, which may be the stopping point for application. In other embodiments where the unitary sheath may not be clear, and/or where the user is not able to see where the penis is within the sheath, the user may stop moving the device toward the base of the penis, just shy of reaching the base, for example. At this point, the device will look like FIG. 8b from the perspective of the wearer.

The user will then transition the retainer section from the rolled position to the unrolled position by either flicking the rolled material 812 of the retainer section 880 toward the base of the penis (as described above), or in embodiments that include a pulley, the user may pull the pulley upward toward the base of the penis to unroll the material of the retainer section (as described above).

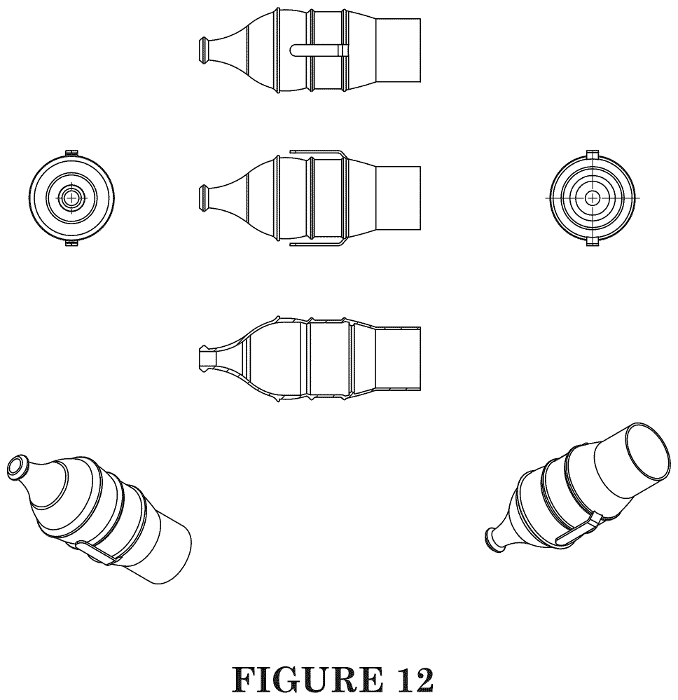

As has been previously discussed, the device may have different sizes to accommodate differently sized appendages, and/or adults and children, for example. However, in some embodiments, a device may have the dimensions provided in FIG. 12, though it will be understood that embodiments of the present disclosure may have any other suitable dimensions.

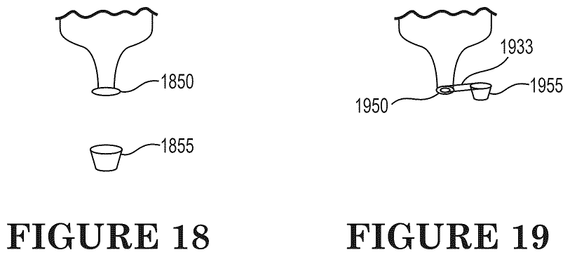

In some embodiments, the device may also be used as a daytime urinary incontinence (UI) device for men who experience involuntary urine leakage (or “urine dribble”), during wake and/or sleep. In some embodiments of a UI device, any embodiment or variation of an embodiment of the catheter described herein may be used. However, the urine exit opening of the catheter device is closed, thereby creating a comfortable, discrete UI device that may be worn during day and/or night to prevent leakage of urine onto the user's clothing, bedding, or anything else the user may contact. The urine exit opening may be closed or sealed in any suitable way or combination of ways. For example, as shown in FIG. 18, a stopper or cork 1855 may be included that may be inserted into the urine exit opening 1850 of the device, which may stop any urine leakage from exiting the device. Such a stopper may be comprised of any suitable material, such as plastic, or any other material or combination of materials. In some embodiments, the stopper may be a duckbill valve. In some embodiments, as shown in FIG. 19, the stopper 1895 may be attached by a lead 1933 to the end of end of the urine exit end 1950 of the sheath, such that when the stopper 1955 is placed in the urine exit opening 1950, the lead 1933 forms a loop. Alternately, a clip, rather than a stopper may be used to seal off the urine exit end. Such a clip could be either free-standing, or attached by a lead to the device. The clip could be made of any suitable material, including, but not limited to plastic and/or metal. In still other embodiments, a small bag may be tied to the urine exit end to close off the urine exit opening. It will be appreciated that any other method, or combination of methods for closing off the urine exit end are also within the spirit and scope of the present disclosure. In still another embodiment, a duckbill valve may be used to seal the urine exit opening.

In other embodiments, a UI device may include all or any of the features described herein, except that the sheath is configured without a urine exit end opening. Instead, the sheath terminates without an opening at the end opposite that of the insertion end.

Users could simply wear the UI device without needing a bag or other accessory to prevent spillage or leakage of urine onto their clothing or other materials or items.

While embodiments of the present disclosure have discussed the collar or grip in the context of an external male catheter, it is to be understood that a collared device may be used in other contexts and is within the spirit and scope of the present disclosure. For example, in some embodiments, the collar may be used for wound care. The collar may be used to hold dressing on, for example, a finger, toe, hand, or other part of the body. The dressing or other material that may be used to cover or enclose a part of the body may be referred to as a sleeve, whereby the sleeve is attached to the collar in the same or similar manner as discussed above with regard to the penis cover. The sleeve may be comprised of any suitable material for the purpose intended, for example, the sleeve may be comprised of cotton or other woven or non-woven absorbent material. The absorbent material may further be covered with a protective layer to prevent the absorbent material from becoming wet, for example, or to prevent the covered body part from being harmed. The protective layer may be comprised of a relatively stiff plastic or other material or combination of materials.

It will be understood that while specific embodiments and exemplary embodiments have been described and disclosed, any of the features disclosed may be included within any embodiment. As such, embodiments are not limited to the description expressly provided, but also include disclosure provided related to other disclosed embodiments and features thereof. Further use of different names for similar portions of a device does not necessarily denote any specific feature being included or excluded simply because of the use of one name for a portion of a device versus another.

What is claimed is:

1. An improved external male catheter, comprising:
a tubular sheath with an opening therethrough, the sheath having an insertion end and a urine tube adapter end, whereby the opening at the urine tube adapter end is sized and shaped to attach to a urine drainage tube; and
a collar comprised of a stiffer material than the tubular sheath, the collar being fixedly attached to the insertion end of the tubular sheath, the collar comprising a ring with an internal circumference and an external circumference, wherein the external circumference is smaller than a circumference of the opening on the tubular sheath at the insertion end.

2. The improved external male catheter of claim 1, wherein the collar is comprised of silicone that substantially holds its shape when pressure is applied to the collar.

3. The improved external male catheter of claim 2, wherein the collar is fixedly attached to the insertion end of the tubular sheath by adhesive tape.

4. The improved external male catheter of claim 2, wherein the collar is fixedly attached to the insertion end of the tubular sheath by a heat-seal adhesive.

5. The improved external male catheter of claim 2, further comprising an internal wrap, the internal wrap comprising a ring, the internal wrap being fixedly attached to the collar, wherein the internal wrap is thread through the internal circumference of the collar and folded over a portion of the external circumference of the collar, wherein, the internal wrap has a length greater than that of the collar.

6. The improved external male catheter of claim 5, wherein the internal wrap is comprised of a thin, flexible material.

7. The improved external male catheter of claim 6, wherein the folded over portion of the internal wrap is adhered to the portion of the external circumference of the collar by tape.

8. The improved external male catheter of claim 6, wherein the folded over portion of the internal wrap is adhered to the portion of the external circumference of the collar by a heat-seal adhesive.

9. The improved external male catheter of claim 2, further comprising a ring-shaped pad with a circumference smaller than the internal circumference of the collar, wherein the ring-shaped pad is fitted inside the internal circumference of the collar.

10. The improved external male catheter of claim 9, wherein the ring-shaped pad is secured to the internal circumference of the collar by an adhesive.

11. The improved external male catheter of claim 2, further comprising a stopper sized and shaped to snugly fit into the opening at the urine tube adapter end of the tubular sheath.

12. A collar for securing to a part of the penis, the collar comprising:
a ring, the ring having an interior circumferential surface and an exterior circumferential surface; and
a tubular-shaped internal wrap attached to the collar by threading the internal wrap through the interior circumferential surface of the collar and folding a portion of the internal wrap over the exterior circumferential surface of the collar.

13. The collar of claim 12, wherein the folded-over portion of the internal wrap is secured to the exterior circumferential surface of the collar by an adhesive.

14. The collar of claim 13, wherein the adhesive is tape.

15. The collar of claim 12, wherein the adhesive is a heat-seal adhesive.

16. The collar of claim 13, wherein the collar is comprised of silicone.

17. The collar of claim 16, wherein the internal wrap is comprised of nitrile.

18. A method of self-catheterizing, the method comprising:
applying lubricant to a portion of a wearer's penis;
grasping an improved catheter by a collar portion of the catheter;
while holding the collar portion of the improved catheter, inserting the head of the penis through an opening at the collar portion of the catheter until the collar portion of the catheter sits at a base of the penis.

19. The method of claim 18, wherein only one hand is used to grasp and hold the collar portion during self-catheterization.

20. The method of claim 18, wherein the collar is comprised of silicone.
